(19) United States
(12) Patent Application Publication
(10) Pub. No.: US 2009/0171285 A1
(43) Pub. Date: Jul. 2, 2009

(54) Safety syringe, syringe plunger processing system and method

(76) Inventor: Sheei-Jin Wang, Wu-Ku Hsiang (TW)

Correspondence Address: BACON & THOMAS, PLLC, 625 SLATERS LANE, FOURTH FLOOR, ALEXANDRIA, VA, 22314-1176, US

(21) Appl. No.: 12/003,808

(22) Filed: Jan. 2, 2008

(51) Int. Cl. A61M 5/50; B29C 45/00

(52) U.S. Cl. 604/110; 264/238

(57) ABSTRACT

A safety syringe includes a plunger for moving a rubber stopper in a barrel to force the medicine out of a needle assembly into the patient's body that has a connector at the front side of a shank thereof to hold a metal hook for hooking the needle assembly so that the needle assembly can be pulled backwards and received inside the barrel after the service of the safety syringe. The invention relates also automatic fabrication of the plunger through a syringe plunger processing system that comprises a metal hook dispenser for dispensing metal hooks, a connector dispenser for dispensing connectors, a high-frequency heat-sealing apparatus for bonding each metal hook to one respective connector, and an injection molding apparatus for molding a shank on each combination of metal hook and connector.

### BACKGROUND OF THE INVENTION

1. Field of the Invention

The present invention relates to a hypodermic syringes and more particularly, to a safety syringe of which a plunger comprises a shank, a connector at the front end of the shank, and a metal hook affixed to the connector for hooking a needle assembly for enabling the needle assembly to be received inside a barrel after the service of the safety syringe. The invention relates also to the fabrication of the plunger.

2. Description of the Related Art

If a syringe is repeatedly used, disease infection will happen on the patient. Therefore, disposable syringes are commonly used for clinical application. An ordinary disposable syringe essentially comprises a barrel, a plunger having a rubber plunger head and a needle assembly. During use, the plunger is pushed forwards, causing the rubber plunger head to squeeze medicine out of the barrel through a needle at a needle hub of the needle assembly into the body of the patient. After use, the nurse or medical staff must cap a tip protector on the front side of the barrel to protect the needle, preventing accidental injury. However, this kind of ordinary disposable syringe is not self-destructive. It is difficult to stop illegal collecting and recycling of waste disposable syringes for repeatedly used. It is dangerous to recycle waste disposable syringes for clinical use. In case waste disposable syringes are repeatedly used, disease infection (AIDS, syphilis, type B hepatitis, type C hepatitis, tuberculosis, Typhoid fever, diphtheria, etc.) will happen on the patient.

There are self-destructive syringes that eliminate any reusing of the assembly to provide safety and reliability. However, the fabrication of regular self-destructive syringes requires much labor and time, resulting in a high manufacturing cost. Further,during manual assembly process, the component parts tend to be contaminated accidentally.

### SUMMARY OF THE INVENTION

The present invention has been accomplished under the circumstances in view. It is therefore the main object of the present invention to provide a safety syringe, which is self-destructive to prevent illegal collecting and recycling and accidental injury. It is another object of the present invention to provide a safety syringe fabrication method, which simplifies the fabrication of safety syringes, saving much time and labor and effectively lower the cost. To achieve these and other objects of the present invention, the safety syringe comprises a barrel, a needle assembly and a plunger. The plunger comprises a shank, a connector at the front side of the shank, and a metal hook bonded to the connector for hooking the needle assembly so that the needle assembly can be pulled backwards and received inside the barrel after the service of the safety syringe. The shank has a breaking neck through which the shank can easily be broken after the use of the safety syringe, leaving the broken front part of the plunger inside the barrel.

To achieve these and other objects of the present invention, the safety syringe fabrication method comprises a syringe plunger fabrication procedure that is performed by means of a syringe plunger processing system. The syringe plunger processing system comprises a metal hook dispenser adapted to dispense metal hooks, a connector dispenser adapted to dispense connectors, a high-frequency heat-sealing apparatus adapted to bond each metal hook to one respective connector, and an injection molding apparatus adapted to mold a shank on each combination of metal hook and connector.

### BRIEF DESCRIPTION OF THE DRAWINGS

FIG. 1 is a block diagram describing the flow of a syringe plunger fabrication method according to the present invention.

FIG. 2 is a schematic top view showing the fabrication of safety plungers in a processing system according to the present invention.

FIG. 3 is an elevational view of a safety syringe according to the present invention (the protective cap opened).

FIG. 4 is an exploded view of the safety syringe according to the present invention.

FIG. 5 is a sectional side view of FIG. 4.

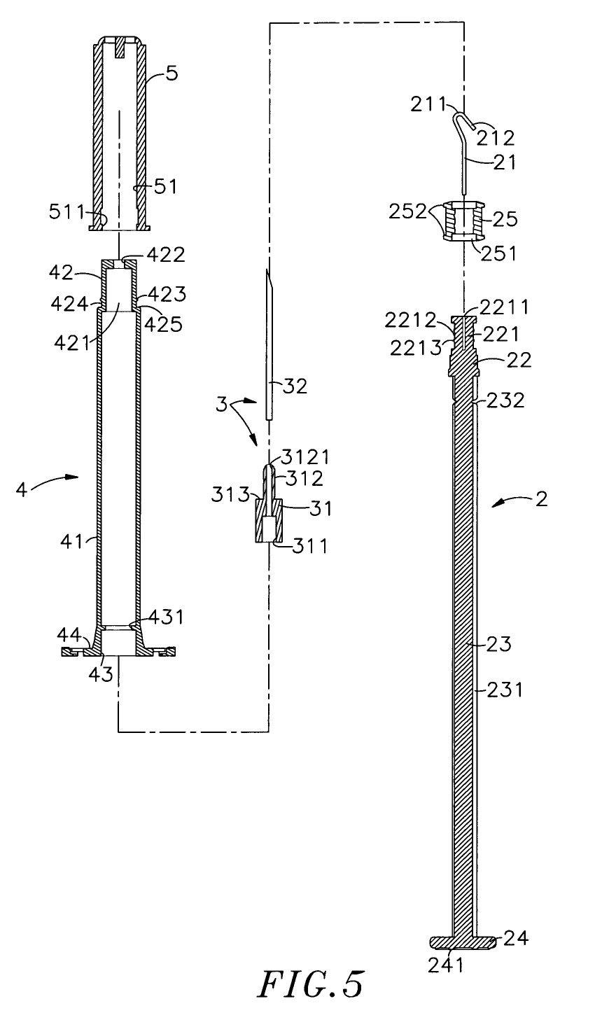

FIG. 6 is a sectional side assembly view of the safety syringe according to the present invention.

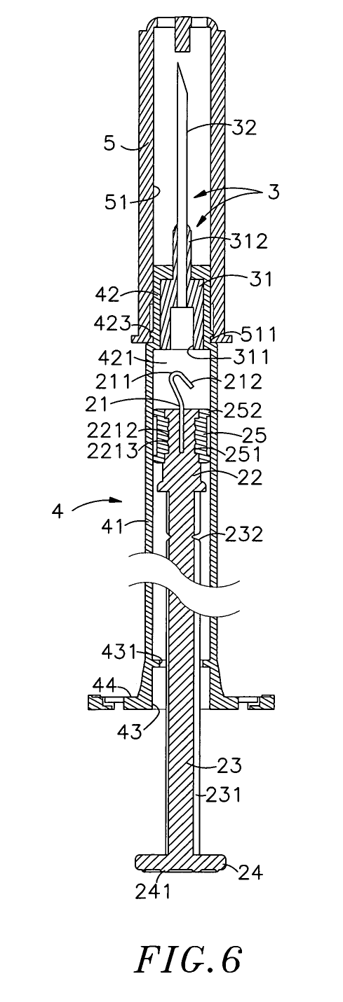

FIG. 7 is a sectional side view of the present invention, showing the metal hook engaged into the bottom receiving hole of the needle hub after injection.

FIG. 8 is a sectional side view of the present invention, showing the needle assembly biased inside the tubular body of the barrel after injection.

FIG. 9 is a schematic sectional side view of the present invention, showing the shank of the plunger broken after injection.

FIG. 10 is an exploded view of an alternate form of the plunger according to the present invention.

### DETAILED DESCRIPTION OF THE PREFERRED EMBODIMENT

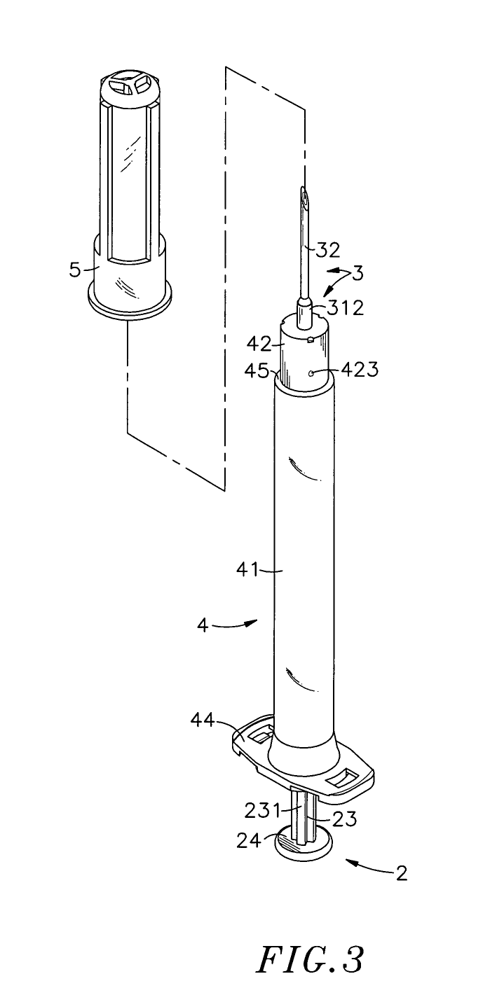

Referring to FIGS. 3˜6, a safety syringe in accordance with the present invention is shown comprised of a plunger 2, a needle assembly 3, a barrel 4 and a protective cap 5.

The plunger 2 comprises a metal hook 21, a connector 22, a shank 23, a thumb rest 24 and a rubber stopper 25. The metal hook 21 has a front end biased at an angle and terminating in a front U-turn 211 and then a hook tip 212. A bottom end of the metal hook 21 is fixedly fastened to the connector 22. The connector 22 comprises an insertion hole 2211 axially extending to the front end, a reduced diameter portion 221, a locating groove 2212 extending around the periphery of the reduced diameter portion 221, and at least one locating flange 2213 extending around the periphery of the locating groove 2212. The bottom end of the metal hook 21 is inserted into the insertion hole 2211 of the connector 22. After insertion of the bottom end of the metal hook 21 into the insertion hole 2211 of the connector 2, the connector 2 is sealed to the metal hook 21 by means of high-frequency heat sealing. The shank 23 comprises a plurality of longitudinal ribs 231 protruded from the periphery and extending along the length between the connector 22 and the thumb rest 24, and a breaking neck 232 near the connector 22. The thumb rest 24 has a plurality of anti-slip blocks 241 at the bottom side. Further, the connector 22 and the thumb rest 24 are respectively and directly molded on the front and rear ends of the shank 23. The rubber stopper 25 is fastened to the reduced diameter portion 221 of the connector 22, having a mounting through hole 251 extending through its front and rear sides, and two deformable stopper flanges 252 extending around the periphery of the front and rear sides and stopped against the inner surface of the barrel 4. The inside wall of the rubber stopper 25 fits the configuration of the reduced diameter portion 221, the locating groove 2212 and the locating flange 2213 of the connector 22 so that the rubber stopper 25 is firmly secured to the connector 22 after insertion of the connector 22 through the mounting through hole 251.

The needle assembly 3 is comprised of a needle hub 31 and a needle 32. The needle hub 31 has a bottom receiving hole 311, a front stop edge 313, a hollow front extension rod 312 forwardly axially extending from the center of the front stop edge 313, and a needle hole 3121 axially extending through the hollow front extension rod 312 in communication with the bottom receiving hole 311. The needle 32 is inserted into the needle hole 3121 and fixedly secured thereto.

The barrel 4 comprises a tubular body 41, a front neck 42 axially forwardly extending from the front end of the tubular body 41, a front receiving chamber 421 defined within the front neck 42 in communication with the inside space of the tubular body 41, a front center through hole 422 axially extending through the front side of the front neck 42, raised stop portions 423 protruded from and arranged around the periphery of the front neck 42, a step 425 connected between the front neck 42 and the tubular body 41, a positioning groove 424 defined around the periphery of the front neck 42 between the step 425 and the raised stop portions 423, an annular inside stop flange 431 extending around the inside wall of the tubular body 41 near a rear opening 43 of the tubular body 41, and a finger flange 44 extending around the periphery of the rear end of the tubular body 41.

The protective cap 5 has a receiving hole 51 axially extending to its rear side, and an annular inside retaining flange 511 extending around the inside wall in the rear side of the receiving hole 51.

During the assembly process of the safety syringe, the needle assembly 3 is inserted through the rear opening 43 of the tubular body 41 of the barrel 4 and fitted into the front receiving chamber 421 of the front neck 42 to force the hollow front extension rod 312 of the needle hub 31 through the front center through hole 422 of the front neck 42 of the barrel 4. At this time, the front stop edge 313 of the needle hub 31 is stopped against the front wall of the front neck 42, and therefore the needle hub 31 is firmly secured in the front receiving chamber 421 of the front neck 42. Thereafter, the metal hook 21 and the connector 22 are inserted with the shank 23 of the plunger 2 through the rear opening 43 into the inside of the tubular body 41 of the barrel 4 to rub the deformable stopper flanges 252 of the rubber stopper 25 over the inside wall of the tubular body 41. After insertion of the plunger 2 into the barrel 4, the thumb rest 24 is suspending outside the barrel 4. Thereafter, the protective cap 5 is capped on the front neck 42 of the barrel 4 to engage the annular inside retaining flange 511 into the positioning groove 424. At this time, the raised stop portion 423 is stopped at the front side of the annular inside retaining flange 511, securing the protective cap 5 to the barrel 4 positively.

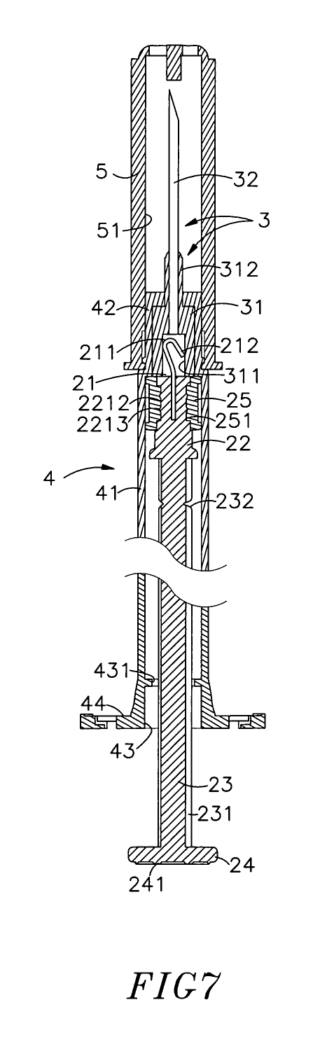

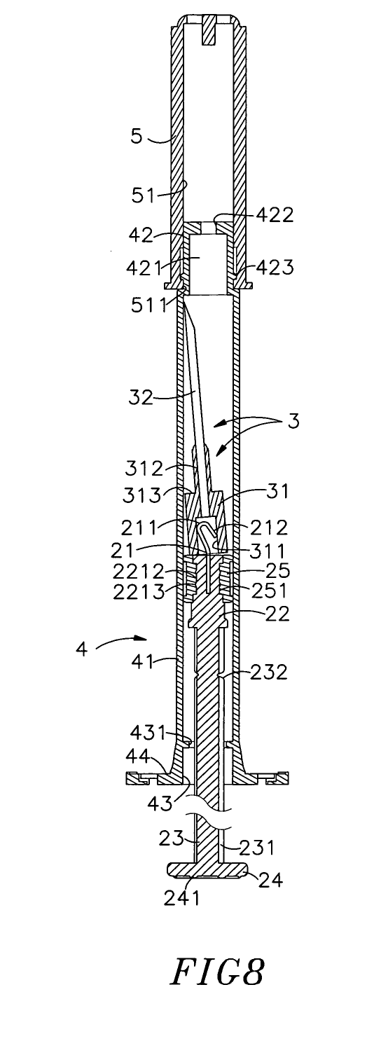

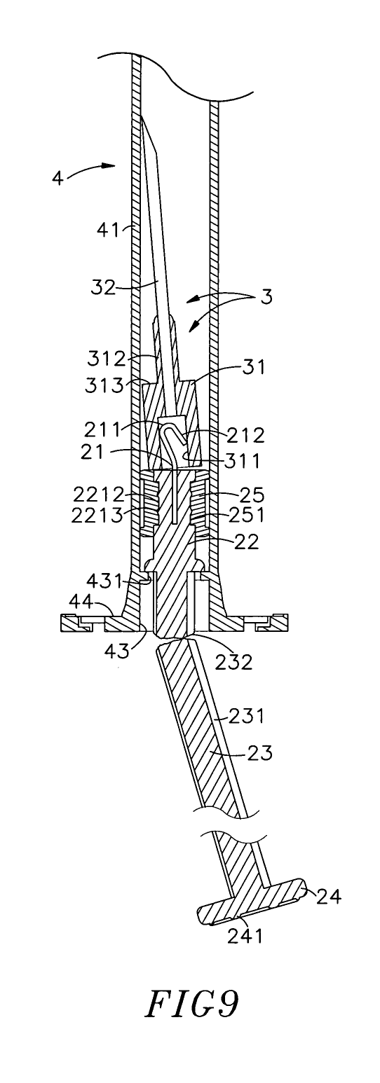

Referring to FIGS. 7˜9, by means of holding the finger flange 44 of the barrel 4 with the fingers and pushing the thumb rest 24 with the thumb, the plunger 2 is moved forwards in the barrel 4, and the deformable stopper flanges 252 of the rubber stopper 25 are rubbed over the inside wall of the tubular body 41 of the barrel 4 to seal the tubular body 41 in an airtight status, and therefore the contained fluid medicine is forced out of the barrel 4 through the bottom receiving hole 311 of the needle hub 31 and the needle 32 into the patients body by the rubber stopper 25 as the rubber stopper 25 is moving forwards with the connector 22 and the shank 23. When the front deformable stopper flange 252 of the rubber stopper 25 is stopped at the step 425 of the barrel 4, the contained fluid medicine is completely forced out of the tubular body 41 of the barrel 4 and at the same time, the metal hook 21 is inserted into the bottom receiving hole 311 of the needle hub 31 to hook the front U-turn 211 and the hook tip 212 in the inside wall of the needle hub 31. Because the front U-turn 211 bias from the metal hook 21 at an angle and the transverse width of front the U-turn 211 is approximately equal to the diameter of the bottom receiving hole 311 of the needle hub 31, the metal hook 21 is affixed to the needle hub 31 after insertion of the front U-turn 211 and the hook tip 212 into the bottom receiving hole 311 of the needle hub 31. After injection, pull the plunger 2 backwards relative to the barrel 4, at this time, the needle assembly 3 is pulled backwards by the metal hook 21 of the plunger 2 into the inside of the tubular body 41 of the barrel 4. When the needle 32 is disconnected from the front neck 42 of the barrel 4 during its backward movement with the needle hub 31 and the plunger 2, the springy power of the front U-turn 211 of the metal hook 21 forces the needle assembly 3 to bias with the front U-turn 211, and therefore the tip of the needle 32 is stopped against the inside wall of the tubular body 41 of the barrel 4. If the plunger 2 is pushed forwards accidentally at this time, the tip of the needle 32 will be stopped against the step 425, and therefore the needle 32 is safety kept inside the barrel 4 after injection, preventing an accidental injury (see FIG. 8). Further, the plunger 2 can be pulled backwards relative to the barrel 4 to the extent that the rear side of the connector 22 is stopped at the annular inside stop flange 431 and then biased to break the breaking neck 232, leaving the front part of the plunger 2 inside the barrel 4 (see FIG. 9).

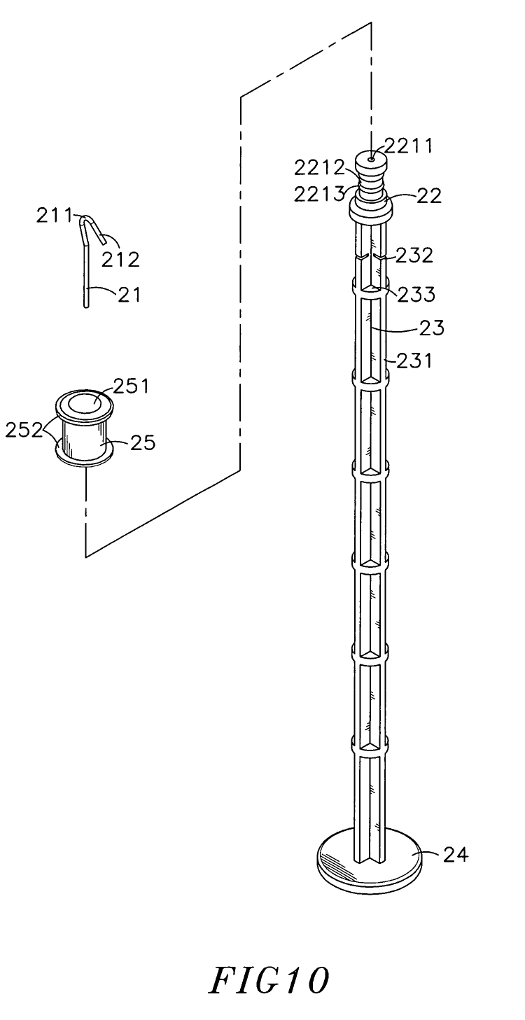

FIG. 10 is an exploded view of an alternate form of the plunger according to the present invention. According to this embodiment, the shank 23 comprises a plurality of longitudinal ribs 231 protruded from the periphery and extending along the length between the connector 22 and the thumb rest 24, a breaking neck 232 near the connector 22, and transverse ribs 233 extending around the periphery at different elevations and joining the longitudinal ribs 231. The transverse ribs 233 reinforce the structural strength of the shank 23, and prevents vibration of the shank 23 when the shank 23 is moved in and out of the tubular body 41 of the barrel 4.

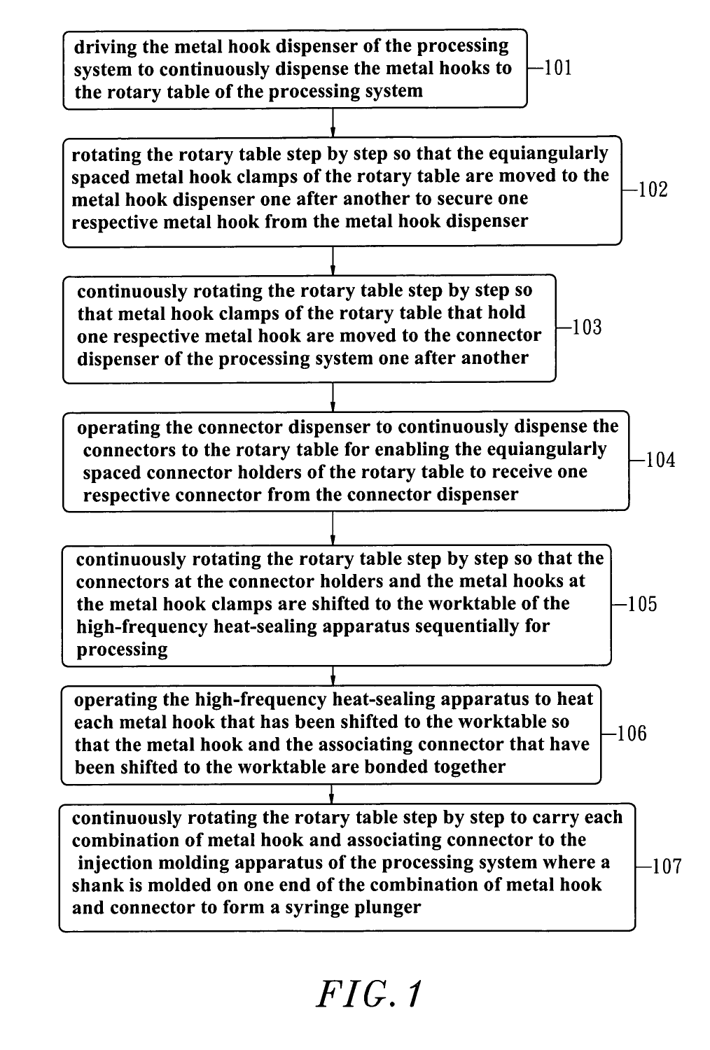

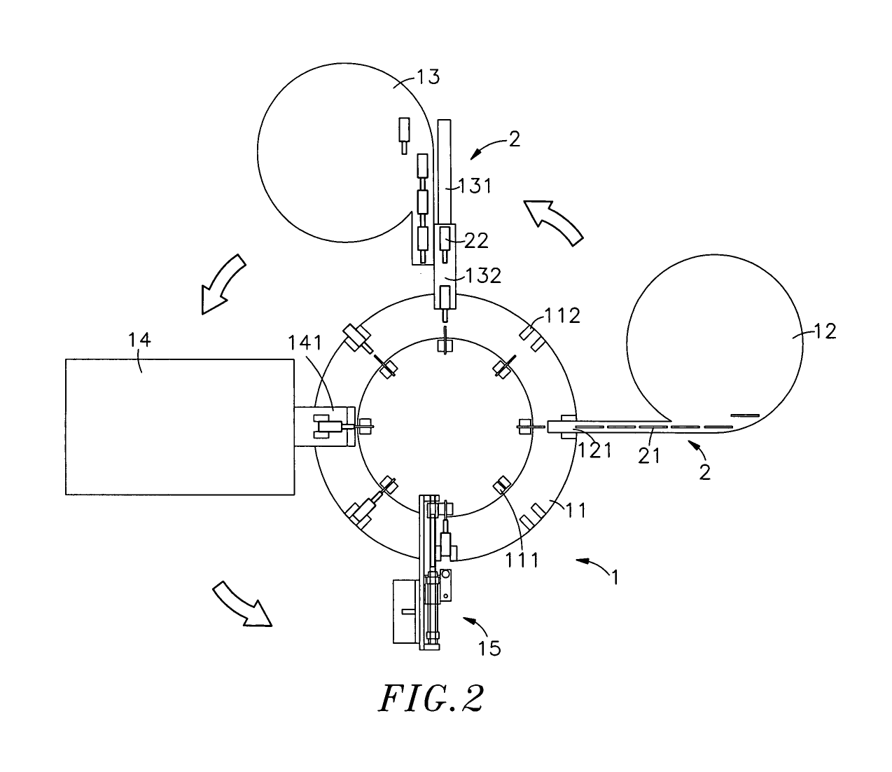

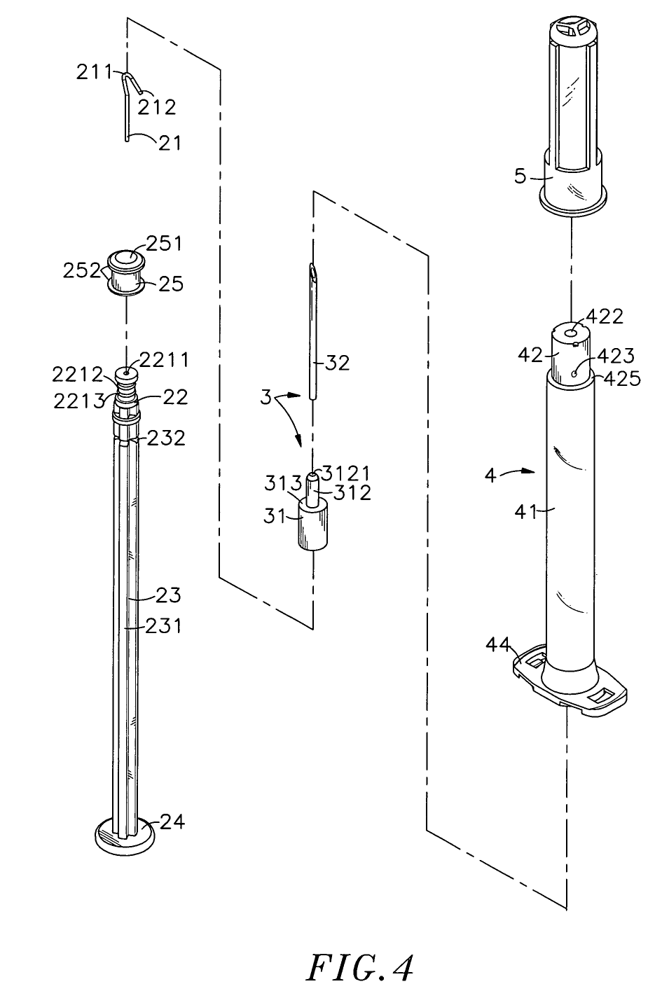

Referring to FIGS. 1 and 2 and also FIGS. 4˜6, a safety syringe fabrication method in accordance with the present invention includes a syringe plunger fabrication procedure for the fabrication of the aforesaid plunger 2 (the rubber stopper 25 excluded). The syringe plunger fabrication procedure is performed through a processing system 1. The processing system 1 comprises a rotary table 11, a metal hook dispenser 12, a connector dispenser 13, a high-frequency heat-sealing apparatus 14 and an injection molding apparatus 15. The metal hook dispenser 12, the connector dispenser 13, the high-frequency heat-sealing apparatus 14 and the injection molding apparatus 15 are equiangularly spaced around the rotary table 11. The rotary table 11 comprises a plurality of equiangularly spaced metal hook clamps 111, and a plurality of equiangularly spaced connector holders 112 corresponding to the metal hook clamps 11.

The syringe plunger fabrication procedure comprises the steps of:
101 driving the metal hook dispenser 12 of the processing system 1 to continuously dispense metal hooks 21 to a rotary table 11 of the processing system 1 through a delivery track 121;
102 rotating the rotary table 11 step by step at a predetermined speed so that the equiangularly spaced metal hook clamps 111 of the rotary table 11 are moved to the metal hook dispenser 12 one after another to secure one respective metal hook 21 from the metal hook dispenser 12;
103 continuously rotating the rotary table 11 step by step so that the metal hook clamps 111 of the rotary table 11 that hold one respective metal hook 21 are moved to the connector dispenser 13 one after another;
104 operating the connector dispenser 13 to continuously dispense connectors 22 to the rotary table 11 through a delivery track 132 for enabling each of the equiangularly spaced connector holders 112 of the rotary table 11 to hold one respective connector 22 in line with the metal hook 21 at the associating metal hook clamp 111;
105 continuously rotating the rotary table 11 step by step so that the connectors 22 at the connector holders 112 and the metal hooks 21 at the metal hook clamps 11 are shifted to a worktable 141 of the high-frequency heat-sealing apparatus 14 sequentially for processing;
106 operating the high-frequency heat-sealing apparatus 14 to heat each metal hook 21 that has been shifted to the worktable 141 so that the metal hook 21 and the associating connector 22 that have been shifted to the worktable 141 are bonded together;
107 continuously rotating the rotary table 11 step by step to carry each combination of metal hook 21 and associating connector 22 to the injection molding apparatus 15 where a shank 23 is molded on one end of the combination of metal hook 21 and connector 22, forming a plunger 2.

During step-by-step rotation of the rotary table 11, the metal hook dispenser 12 of the processing system 1 is operated to continuously dispense metal hooks 21 to the rotary table 11 of the processing system 1 through the delivery track 121 in such a manner that one metal hook 21 is secured to each metal hook clamp 111 of the rotary table 11. After having secured one metal hook 21 from the metal hook dispenser 12, each metal hook clamp 111 and the associating connector holder 112 are shifted to the next processing workstation, i.e., the connector dispenser 13 where a reciprocating pusher 131 pushes one connector 22 out of the delivery track 132 of the connector dispenser 13 into the connector holder 112 of the rotary table 11 that reached the connector dispenser 13. The reciprocating pusher 131 can be a hydraulic cylinder, pneumatic cylinder, slide bearing, sliding rail, or any of a variety of other reciprocating mechanisms. After having secured on connector 22 from the connector dispenser 13, each connector holder 112 is shifted to the next processing station, i.e., the high-frequency heat-sealing apparatus 14 where electric current goes through a high-frequency coil, causing the metal hook 21 at the metal hook clamp 111 that reached the worktable 141 of the high-frequency heat-sealing apparatus 14 and forced into the insertion hole 2211 of the connector 22 at the associating connector holder 112, and therefore the connector 22 is bonded to the associating metal hook 21. After bonding, the combination of metal hook 21 and connector 22 at each metal hook clamp 111 and each associating connector holder 112 is shifted to the next processing station, i.e., the injection molding apparatus 15 where a shank 23 is molded on one end of the combination of metal hook 21 and connector 22, forming a plunger 2.

By means of the aforesaid plunger fabrication procedure, the fabrication of safety syringe is simplified, saving much labor and time.

The safety syringe and its fabrication method according to the present invention has the following features and advantages:

1. By means of the high-frequency heat-sealing apparatus 14, each metal hook 21 and the associating connector 22 are rapidly and fixedly bonded together.

2. By means of the processing system 1, the fabrication of the safety syringe is simple and efficient, saving much labor and time and assuring high quality.

3. When the plunger 2 is inserted into the tubular body 41 of the barrel 4, the deformable stopper flanges 252 of the rubber stopper 25 are rubbed over the inside wall of the tubular body 41, and the tubular body 41 is maintained in an airtight status.

4. When the rubber stopper 25 is being moved forwards with the connector 22 and the shank 23 relative to the barrel 4, the deformable stopper flanges 252 of the rubber stopper 25 are rubbed over the inside wall of the tubular body 41 to maintain the tubular body 41 in an airtight status, and the contained fluid medicine is completely forced out of the barrel 4 through the bottom receiving hole 311 of the needle hub 31 and the needle 32 into the patient's body.

5. When the contained fluid medicine is completely forced out of the barrel 4 through the bottom receiving hole 311 of the needle hub 31 and the needle 32 into the patient's body, the front U-turn 211 and the hook tip 212 of the metal hook 21 are forced into the bottom receiving hole 311 of the needle hub 31, and therefore the metal hook 21 is firmly secured to the needle hub 31.

6. When pulling the plunger 2 backwards relative to the barrel 4 after injection, the needle assembly 3 is pulled backwards by the metal hook 21 of the plunger 2 into the inside of the tubular body 41 of the barrel 4. When the needle 32 is disconnected from the front neck 42 of the barrel 4 during its backward movement with the needle hub 31 and the plunger 2, the springy power of the front U-turn 211 of the metal hook 21 forces the needle assembly 3 to bias with the U-turn 211, and therefore the tip of the needle 32 is stopped against the inside wall of the tubular body 41 of the barrel 4.

7. If the plunger 2 is pushed forwards accidentally after injection, the tip of the needle 32 will be stopped against the step 425, and therefore the needle 32 is safety kept inside the barrel 4 after injection, preventing an accidental injury.

8. After pulled the plunger 2 backwards relative to the barrel 4 to the extent that the rear side of the connector 22 is stopped at the annular inside stop flange 431, the shank 23 is biased relative to the barrel 4 to break the breaking neck 232, leaving the front part of the plunger 2 inside the barrel 4.

9. The shank 23 of the plunger 2 can be made having transverse ribs 233 extending around the periphery at different elevations and joining the longitudinal ribs 231. The transverse ribs 233 reinforce the structural strength of the shank 23, and prevents vibration of the shank 23 when the shank 23 is moved in and out of the tubular body 41 of the barrel 4.

Although particular embodiments of the invention has been described in detail for purposes of illustration, various modifications and enhancements may be made without departing from the spirit and scope of the invention. Accordingly, the invention is not to be limited except as by the appended claims.

What the invention claimed is:

1. A syringe plunger fabrication method comprising the steps of:
(a) driving a metal hook dispenser of a processing system to continuously dispense metal hooks to a rotary table of the processing system;
(b) rotating the rotary table step by step at a predetermined speed so that equiangularly spaced metal hook clamps of the rotary table are moved to the metal hook dispenser one after another to secure one respective metal hook from a delivery track of the metal hook dispenser;
(c) continuously rotating the rotary table step by step so that the metal hook clamps of the rotary table that hold one respective metal hook are moved to a connector dispenser of the processing system one after another;
(d) operating the connector dispenser to continuously dispense connectors to the rotary table for enabling equiangularly spaced connector holders of the rotary table to receive one respective connector from a delivery track of the connector dispenser;
(e) continuously rotating the rotary table step by step so that the connectors at the connector holders and the metal hooks at the metal hook clamps are shifted to a worktable of a high-frequency heat-sealing apparatus sequentially for processing;
(f) operating the high-frequency heat-sealing apparatus to heat each metal hook that has been shifted to the worktable so that the metal hook and the associating connector that have been shifted to the worktable are bonded together; and
(g) continuously rotating the rotary table step by step to carry each combination of metal hook and associating connector to an injection molding apparatus of the processing system where a shank is molded on one end of the combination of metal hook and connector to form a syringe plunger.

2. The syringe plunger fabrication method as claimed in claim 1, comprising a rotary table, said rotary table comprising a plurality of equiangularly spaced metal hook clamps and a plurality of equiangularly spaced connector holders corresponding to said metal hook clamps, a metal hook dispenser adapted to dispense metal hooks to said metal hook clamps of said rotary table, a connector dispenser adapted to dispense connectors to said connector holders of said rotary table, a high-frequency heat-sealing apparatus adapted to seal each metal hook clamped by each of said metal hook clamps to each connector held by each of said connector holders respectively to form a respective combination of metal hook and connector, and an injection molding apparatus adapted to injection-mold a shank onto each combination of metal hook and connector carried on said rotary table.

3. The syringe plunger fabrication method as claimed in claim 1, wherein said connector dispenser comprises a reciprocating pusher adapted to push one connector into each connector holder of said rotary table through said delivery track, said reciprocating pusher being selectively formed of a hydraulic cylinder, a pneumatic cylinder, a slide bearing and a sliding rail.

4. The syringe plunger fabrication method as claimed in claim 1, wherein said high-frequency heat-sealing apparatus is controllable to force said metal hook being held by each metal hook clamp of said rotary table into an insertion hole of said connector being held by said associating connector holder of said rotary table when said high-frequency heat-sealing apparatus is heating said respective metal hook.

5. A safety syringe comprising a barrel, a needle assembly formed of a needle hub and a needle, a plunger, a rubber stopper mounted on said plunger and movable with said plunger in said barrel to force a fluid out of said barrel, and a protective cap for capping on said barrel to protect said needle, wherein:
said barrel comprises a tubular body, a front neck axially forwardly extending from a front end of said tubular body, a front receiving chamber defined within said front neck in communication with the inside space of said tubular body, and a front center through hole axially extending through a front side of said front neck;
said needle hub comprises a bottom receiving hole, and a hollow front extension rod for holding said needle in communication with said bottom receiving hole;
said plunger comprises a shank, a connector fixedly connected with a front end of said shank, said connector having a reduced diameter portion to hold said rubber stopper around the periphery of said connector for enabling said rubber stopper to be moved with said plunger relative to said tubular body of said barrel, and a metal hook fixedly fastened to a front side of said connector for engaging into said bottom receiving hole of said needle hub so that said needle and said needle hub are movable with said plunger to the inside of said tubular body of said barrel when said plunger is pulled backwards after the service of the safety syringe, said metal hook having a front U-turn biased at an angle and terminating in a hook tip for hooking said needle hub;
said protective cap comprises a receiving hole for receiving said front neck of said barrel.

6. The safety syringe as claimed in claim 5, wherein said front neck of said barrel comprises a plurality of raised stop portions protruded from and arranged around the periphery, and a positioning groove defined around the periphery adjacent to said tubular body; said protective cap comprises an annular inside retaining flange extending around an inside wall in a rear side of said receiving hole thereof that is engaged into the positioning groove of said front neck of said barrel when said protective cap is capped on said front neck of said barrel.

7. The safety syringe as claimed in claim 5, wherein said barrel comprises a step connected between said front neck and said tubular body, an annular inside stop flange extending around an inside wall of said tubular body near a rear opening of said tubular body, and a finger flange extending around the periphery of a rear end of said tubular body.

8. The safety syringe as claimed in claim 5, wherein said needle hub has a needle hole axially extending through said hollow front extension rod in communication with said bottom receiving hole for accommodating said needle.

9. The safety syringe as claimed in claim 5, wherein said needle hub has a front stop edge for enabling said needle hub to be stopped against a front wall of said front neck and firmly secured in said front receiving chamber of said front neck.

10. The safety syringe as claimed in claim 5, wherein said connector of said plunger has an axially extending insertion hole for the mounting of said metal hook, and a locating groove and at least one locating flange extending around the periphery of said reduced diameter portion for securing said rubber stopper.

11. The safety syringe as claimed in claim 10, wherein said rubber stopper has a mounting through hole fitting the configuration of said reduced diameter portion and said locating groove and said at least one locating flange of said connector.

12. The safety syringe as claimed in claim 5, wherein said rubber stopper comprises a plurality of deformable stopper flanges extending around the periphery and stopped against the inside wall of said tubular body of said barrel.

13. The safety syringe as claimed in claim 5, wherein said shank of said plunger comprises a front end fixedly connected to said connector, a rear end fixedly provided with a thumb rest, a plurality of longitudinal ribs protruded from the periphery thereof and extending along the length between said connector and said thumb rest, and a breaking neck disposed near said connector.
